(19) United States
(12) Patent Application Publication
(10) Pub. No.: US 2018/0344204 A1
(43) Pub. Date: Dec. 6, 2018

(54) FEATURES FOR OPTICAL SHAPE SENSE ENABLED DEVICE IDENTIFICATION

(71) Applicant: KONINKLIJKE PHILIPS N.V., EINDHOVEN (NL)

(72) Inventors: Molly Lara Flexman, Melrose, MA (US); Sander Hans Denissen, Best (NL); Wilhelmus Henrica Gerarda Maria VAN DEN BOOMEN, Valkenswaard (NL); Neriman Nicoletta KAHYA, Eindhoven (NL)

(73) Assignee: KONINKLIJKE PHILIPS N.V., EINDHOVEN (NL)

(21) Appl. No.: 15/778,052

(22) PCT Filed: Nov. 11, 2016
(86) PCT No.: PCT/IB2016/056784

§ 371 Date: May 22, 2018

Related U.S. Application Data
(60) Provisional application No. 62/265,546, filed on Dec. 10, 2015.

(51) Int. Cl. A61B 5/06; A61B 5/00

(52) U.S. Cl. CPC A61B 5/065; A61B 2034/2061; A61B 5/7475

(57) ABSTRACT

A system for generating a manual input on a shape sensing fiber includes a shape enabled device (102) including one or more shape sensing optical fibers. An input device (106) is configured on a portion of the one or more shape sensing optical fibers, wherein a change in optical shape sensing data associated with the input device distinguishable from other shape sensing data, generates an input signal. A processor system (112) is configured to receive the input signal and perform an action responsive to the input signal.

### BACKGROUND

### Technical Field

This disclosure relates to medical instruments and more particularly to identification of an active device with shape sensing optical fibers in medical applications.

### Description of the Related Art

Optical shape sensing (OSS) or Fiber-Optical RealShape™ (also known as “Optical Shape Sensing”, “Fiber Shape Sensing”, “Fiber Optical 3D Shape Sensing”, “Fiber Optic Shape Sensing and Localization” or the like) employs light along a multicore optical fiber for device localization and navigation during endovascular intervention. One principle involved makes use of distributed strain measurement in the optical fiber using characteristic Rayleigh backscatter or controlled grating patterns. Multiple optical fibers can be used together to reconstruct a 3D shape, or a single optical fiber with multiple cores that may also be helixed for a lower-profile sensor. The shape along the optical fiber begins at a specific point along the sensor, known as the launch or z=0, and the subsequent shape position and orientation are relative to that point. Optical shape sensing fibers can be integrated into medical devices to provide live guidance of the devices during minimally invasive procedures.

In many procedures, it is often difficult for a user to map a proximal section of a medical device that is visible outside the body to a representative image of the device on a display screen. One way that doctors currently perform this mapping is by moving the device from the proximal section. Then, they can see on the screen which of the devices is moving. However, this is not clinically optimal. The devices have been carefully navigated into position and moving them can cause incorrect positioning and unnecessary trauma to vessels and other tissues. This also wastes time during the procedure.

### SUMMARY

In accordance with the present principles, a system for generating a manual input on a shape sensing fiber includes a shape enabled device including one or more shape sensing optical fibers. An input device is configured on a portion of the one or more shape sensing optical fibers, wherein a change in optical shape sensing data associated with the input device distinguishable from other shape sensing data, generates an input signal. A processor system is configured to receive the input signal and perform an action responsive to the input signal.

Another system for generating a manual input on a shape sensing fiber includes a processor and memory coupled to the processor. The memory includes an optical sensing module configured to interpret optical signals from one or more shape sensing optical fibers, the optical signals including shape sensing data and an input signal generated by a user by changing the one or more shape sensing optical fibers. An input device is configured on a portion of the one or more shape sensing optical fibers, and configured to cause a change in optical shape sensing data associated with the input device which is distinguishable from other shape sensing data, to generate the input signal. The processor and memory are configured to receive the input signal and perform an action responsive to the input signal.

A method for generating a manual input on a shape sensing fiber includes inserting a shape enabled device including one or more shape sensing optical fibers into a volume; triggering a change in an input device to generate an input signal, the input device being configured on a portion of the one or more shape sensing optical fibers, wherein a change in the input device is distinguishable from other shape sensing data; and performing an action responsive to the input signal.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a shape sensing system configured to generate an input signal based upon a mechanical change to the fiber and showing a feedback device in accordance with one embodiment;

FIG. 2 is a schematic diagram showing a body having two optical shape sensing devices coupled to a launch fixture where the optical shape sensing devices include input devices in the form of colored bands and feedback indicators in the form of light emitting diodes in accordance with one embodiment;

FIG. 3A is a diagram showing colored bands on an optical fiber to indicate a position where an input may be generated in accordance with one embodiment;

FIG. 3B is a diagram showing textured bands on an optical fiber to indicate a position where an input may be generated in accordance with one embodiment;

FIG. 3C is a diagram showing different stiffness bands on an optical fiber to indicate a position where an input may be generated in accordance with one embodiment;

FIG. 3D is a diagram showing a mechanical device or clip on an optical fiber to indicate a position where an input may be generated and/or to control deflection of the optical fiber in accordance with one embodiment;

FIG. 3E is a diagram showing a ruler banding on an optical fiber to indicate and measure movement of an optical fiber in accordance with one embodiment; and

FIG. 4 is a block/flow diagram showing a method for method for generating a manual input on a shape sensing fiber in accordance with illustrative embodiments.

### DETAILED DESCRIPTION OF EMBODIMENTS

In accordance with the present principles, systems, devices and methods are provided to generate input for devices equipped with fiber optic shape sensing. The present principles provide embodiments where a shape sense enabled device can be employed to generate user input through the shape sensing system. In one useful embodiment, an optical shape sensing fiber or system is integrated within a device to provide three-dimensional (3D) information about a shape and/or pose of the device as well as receive user input to a software application to designate the active status for a particular device or provide a command or trigger for another action. In one embodiment, the input may be employed to distinguish an active device from other devices during a procedure. In particularly useful embodiments, devices may include a trigger mechanism that can cause a local curvature, axial strain, or shape change to be employed to indicate the input or action to the software. For example, the fiber can be integrated into a ‘button’ on the device and when the button is pressed the fiber deforms to provide the input signal. The trigger input may also indicate, for example, to change to display views, highlighting the active device on a display screen, altering controls or menus, mapping a proximal section of the device to an image, etc.

When multiple shape-sensed devices are employed in a procedure, it can be difficult to identify which device physically corresponds to a visualization shown to an operator. A Fiber-Optical RealShape™ (FORS) may be employed to provide ways for identifying the device while in clinical use. If an optical shape sensing fiber is already embedded or attached to a medical instrument for tracking the shape or position of the instrument, the sensor can also be used to provide user input to the software to identify the active device. Different regions of the device can be employed for different types of input. Other features include, e.g., passive visual or haptic banding on the proximal section of the device to indicate fiber input locations; mechanical (e.g., vibration) or acoustic feedback to indicate when a given trigger location has been activated and active visual feedback indicators (e.g., light emitting diodes (LED), on-screen images or other light sources) to indicate when a given trigger location has been activated. Feedback indicators may include acoustic feedback, haptic feedback, visual feedback, etc.

In endovascular aneurysm repair (EVAR), the position of an endograft or stent needs to be known so that other catheters and endografts can be navigated with respect to an original endograft. If the endografts are not correctly positioned, a number of issues may arise. Positioning instruments and identifying which instruments are active is a consideration during a procedure.

Under x-ray guidance, the stent can be visualized through x-ray visible markers that are located in key positions on the stent. In the fenestrated stent, the markers identify the locations of the fenestrations and can be used to orient the stent to appropriately align the fenestrations with the side vessels. In accordance with the present principles, devices and methods provide indications associated with medical instruments during a procedure (e.g., EVAR or fenestrated EVAR (FEVAR)) to visualize the device or devices in displays. In useful embodiments, devices and methods make use of a proximal hub, bands or a trigger mechanism on an optical fiber to provide an input signal to perform an action. The action may include, e.g., indicating in a display image, the device associated with the hub or trigger mechanism that was activated. The hub or trigger mechanism may include a shape profile that deflects the fiber passing through it into a known shape. That shape can be detected along the fiber to show which instrument is being “pinged” and that instrument may be rendered more clearly or distinguished in the display image. This can be applied to many devices such as vascular devices (e.g., catheters, sheaths, deployment systems, etc.), endoluminal devices (e.g., endoscopes), orthopedic devices (e.g., k-wires & screwdrivers) as well as for non-medical devices.

To provide a more efficient registration, a deformable device utilizing Fiber-Optical Real Shape™ (FORS™ also known as “Optical Shape Sensing”, “Fiber Shape Sensing”, “Fiber Optical 3D Shape Sensing”, “Fiber Optic Shape Sensing and Localization” or the like) may be employed. As used herein, the terms FORS™ and FORS™ systems are not, however, limited to products and systems of Koninklijke Philips, N.V., but refer generally to fiber optic shape sensing and fiber optic shape sensing systems, fiber optic 3D shape sensing, fiber optic 3D shape sensing systems, fiber optic shape sensing and localization and similar technologies.

It should be understood that the present invention will be described in terms of medical instruments; however, the teachings of the present invention are much broader and are applicable to any fiber optic instruments. In some embodiments, the present principles are employed in tracking or analyzing complex biological or mechanical systems. In particular, the present principles are applicable to internal tracking procedures of biological systems and procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

It will also be understood that when an element such as a layer, region or material is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

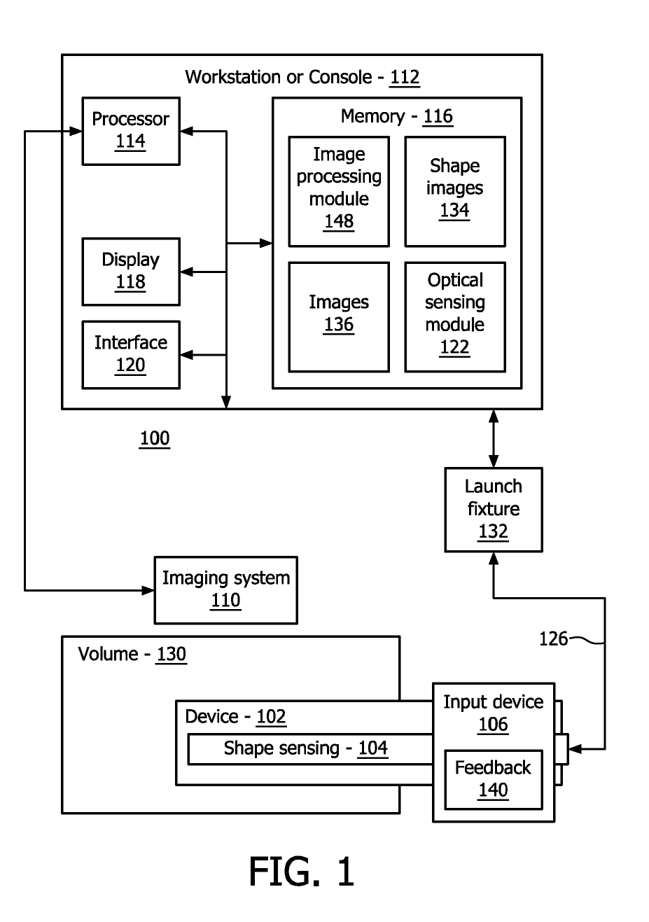

Referring now to the drawings in which like numerals represent the same or similar elements and initially to FIG. 1, a system 100 for monitoring shape sensing enabled devices and other devices is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store an optical sensing module 122 configured to interpret optical feedback signals from a shape sensing device or system 104 (FORS™). Optical sensing module 122 is configured to use the optical signal feedback (and any other feedback) to reconstruct deformations, deflections and other changes associated with shape sensed devices. In accordance with the present principles, a medical device or instrument 102 includes an elongated flexible instrument. The device 102 is configured to receive the FORS™ system 104 therethrough. The medical device 102 may include a catheter, a sheath, a probe, an endoscope, a robot, an electrode, a filter device, a balloon device, a graft, a stent or other medical component, etc. The medical device 102 may include an input device 106, e.g., a hub, banding or trigger mechanism, that may be configured within the device 102, applied (connected/coupled) to the device 102, configured to fit within the device 102 or otherwise access a portion of an optical fiber of fiber 126 of the FORS™ system 104. Other configurations may include a launch fixture 132 between the input device 106 and the shape sensing device 104.

The FORS™ system 104 includes one or more optical fibers 126 which may be arranged in a set pattern or patterns. The optical fibers 126 connect to the workstation 112 through cabling. The cabling may include the fiber optics 126 as well as other connections, e.g., electrical connections, other instrumentation, etc., as needed.

System 104 with fiber optics may be based on fiber optic Bragg grating sensors, Rayleigh scattering, or other types of scattering. Inherent backscatter in conventional optical fiber can be exploited, such as Raleigh, Raman, Brillouin or fluorescence scattering. One such approach is to use Rayleigh scatter in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, or in multiple single-core fibers arranged together, the 3D shape and dynamics of the surface of interest can be followed.

A fiber optic Bragg grating (FBG) system may also be employed for system 104. An FBG is a segment of optical fiber that reflects particular wavelengths of light and transmits all others. This is achieved by adding a periodic variation of the refractive index in the fiber core, which generates a wavelength-specific dielectric mirror. An FBG can therefore be used as an inline optical filter to block certain wavelengths, or as a wavelength-specific reflector. Fresnel reflection at each of the interfaces where the refractive index is changing is measured. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors.

Incorporating three or more cores permits a three dimensional form of such a structure to be precisely determined. From the strain measurement, the curvature of the structure can be inferred at that position. From the multitude of measured positions, the total three-dimensional form is determined. A similar technique can be employed for multiple single-core fibers configured in a known structure or geometry.

In one embodiment, workstation 112 is configured to receive feedback from the shape sensing device 104 in the form of position of shape sensed data as to where the sensing device 104 has been within a volume 130. The shape sensing information or data within the space or volume 130 can be displayed on a display device 118. The shape sensing information or data may be stored as shape images 134.

Workstation 112 includes the display 118 for viewing internal images of a subject (patient) or volume 130 and may include the shape images 134 as an overlay on medical images 136 (of the body or volume 130) such as x-ray images, computed tomography (CT) images, magnetic resonance images (MRI), real-time internal video images or other images as collected by an imaging system 110 in advance or concurrently. Display 118 may also permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112.

The device 102 is visualized in the image or images 136 which may be rendered on the display 118. The device 102 is visualized using the optical shape sensing data. In one embodiment, the device 102 may be attached to the input device 106 at a proximal portion of the device 102. The input device 106 may include a hub or other mechanism where the fiber can be repeatably deformed to generate an input signal. The hub or other mechanism may include a spring-loaded button that deflects the fiber in a distinguishable way to generate a recognizable shape sensed signal in the workstation 112. In other embodiments, the input device 106 may include a series of bands, e.g., colored, textures, stiff/soft, etc. to designate a region where if the fiber is bent an input signal is generated.

To create a meaningful visualization of the device 102, the input device 106 may be mapped to a portion of the device 102 so that an input signal can be distinguished from shape sensing data by the system 100. Examples may include a location at proximal end portion, which can be reference from a reference point of the device 102 or the body, etc. The input signal generated by the input device 106 is distinguishable by the optical sensing module 122 from other shape sensing data. This may be based on a location of the input device 106 relative to the shape sensing system 104 or the shape of a bend of the fiber employed as the input signal. It should be understood that a trigger to generate the input signal from the input device 106 may include any change in the input device 106, e.g., any shape parameter change including, e.g., geometry (x, y, z, twist), axial strain, temperature, curvature, a dynamic pattern (vibration), etc.

The mapping can be done in a plurality of ways. For example, an image processing module 148 may employ a set of nodes or positions along the fiber designated for input, if a change occurs in this region it is interpreted as an input signal. The manner and the shapes of the input signal may be mapped to a command or action so that a plurality of inputs can be understood by the system. Other ways of mapping the device 102 to the input device 106 may include bending the optical fiber associated with the device 102 in a known way (distinguishable by the optical sensing module 122) in the optical shape data. The input device 106 may include a template or fiber-bending mechanism that bends the fiber in a distinctive way that may be visualized in the display image of the shape data, or, automatically detected and used to create some other feedback to the user.

When employing multiple FORS™ devices for a procedure, it is difficult to distinguish which proximal section (outside the body) relates to which device that is visualized inside the body. The present principles provide an input method for clearly distinguishing between the FORS™ devices in an image. The input device 106 may include color or texturing banding, LED indicators, fiber bending mechanisms, etc. in a proximal section for user input. Feedback indicators 140 may be included to provide an indication that the input signal has been received by the system 100. The feedback indicators 140 may include, e.g., acoustic, vibration, haptic, mechanical, optical, etc. indicators. The feedback indicators 140 may also be located on a launch fixture 132 (e.g., LEDs), on the display screen 118, on speakers (interface 120), on the workstation 112, etc.

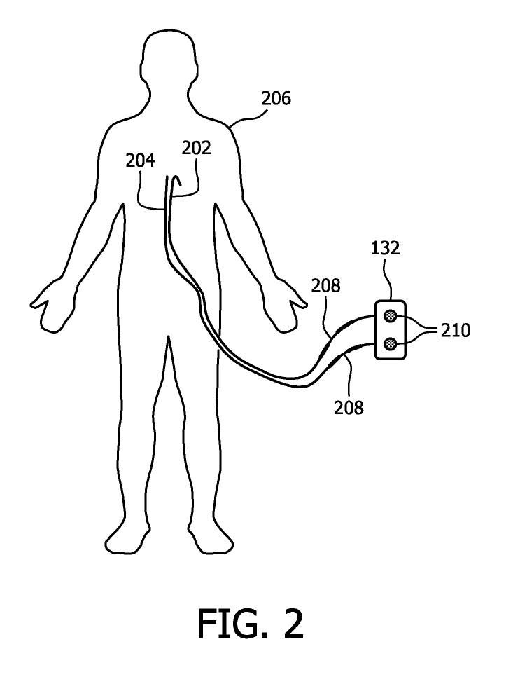

Referring to FIG. 2, FORS-devices 202, 204 employed in an endovascular procedure are illustratively shown in accordance with one illustrative embodiment. Since it is usually difficult to distinguish which proximal section of the devices 202, 204 (outside a body 206) relates to which device 202, 204 that is visualized inside the body 206 by an imaging system or rendered shape change data. Feedback indicators 210 are employed. An input device 208 includes color banding in a proximal section for user input. Feedback indicators 210 include LEDs 210 on the launch fixture 132.

An optical shape sensing fiber within devices 202, 204 can be used for navigation of the interventional devices 202, 204. If an optical shape sensing fiber is already embedded or attached to devices 202, 204 for tracking the shape or position or the devices 202, 204, the optical shape sensing fiber or sensor can also be employed to provide user input to visualization software (148, FIG. 1). For example, if the optical shape sensing in devices 202, 204 includes a designated region (input devices 208), the region can be altered to provide a distinct input signal to the workstation (112, FIG. 1) or the visualization software (148, FIG. 1) to trigger the performance of a task.

The task may include any number of actions including, e.g., highlighting the shape data rendered in a display in a different color or texture, turning an image of the shape data on, changing a visual effect related to the shape data for that fiber (e.g., blinking or increased brightness), etc. The visual effects may be overlaid on an image of the body 206 through the visualization software 148.

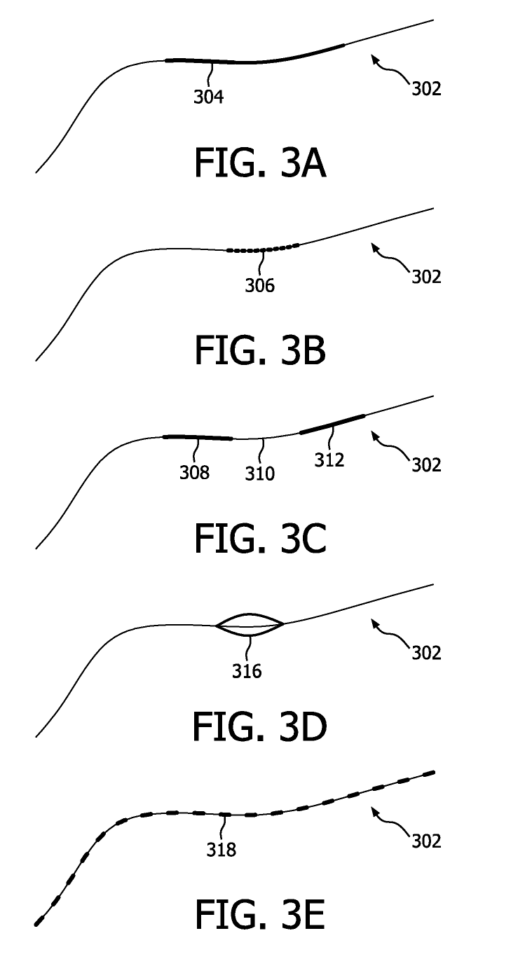

Referring to FIGS. 3A-3E with continued reference to FIGS. 1 and 2, the devices 202, 204 may include a number of features to provide indicators for locations for inputs on the FORS™ devices 202, 204. In one embodiment, visual or haptic markings 304 may be placed on a proximal end portion 302 of the device 202 or 204. These may include, e.g., color bands 304 to indicate fiber input locations as depicted in FIG. 3A. A user may by using their hand (or a tool, such as input device 106) create a small bend in the device 202, in the region of the band. The small bend at the location of the bands or bands 304 may indicate an input to the system. In FIG. 3B, a textured band or bands 306 may be employed instead of (or in addition to) color bands.

The colored 304 or textured bands 306 may be employed with or be employed separately from regions of different stiffness. Regions of different stiffness may include, e.g., a stiff region 308, a soft region 310 and a stiff regions 312. Other embodiments may include two or more alternating stiff and soft regions in any combination. Different stiffnesses may be employed so that the bands are easier to bend at a location where the different areas of stiffness interface each other. This makes the bend more localized.

The colored, textured or stiffness banding can be done in a portion of the device that does not enter the body, e.g., the proximal end portion 302. Different bands of the device can be employed for different types of input. The bands could be integrated into the device itself. The bands may be attached to the device (302) by the user and then the software (148) would employ a calibration step where the user presses each band and connects the band deformation with a given action in the software. In one embodiment, as shown in FIG. 3D, mechanical devices 316 may be placed on the FORS™ device (302) to guide bending. The mechanical devices 316 may include a clip, a spring loaded deflection device (hub) to bend the fiber in a repeatable configuration, a bending mechanism, etc.

The mechanical devices 316 may indicate a position where input is permitted, but also provide mechanical feedback (e.g., vibration, temperature) as the feedback device 140 (FIG. 1), which may be employed to indicate that an input signal has been received by the system by bending the fiber. The vibration could indicate when a given trigger location has been activated. The vibration may be through feedback 140 on the device 102. The vibration may also be used to identify a trigger location (e.g., when the fiber is bent, a vibration is indicated so that the user knows where the input signal can be sent by bending the fiber). The vibration can also be employed to indicate that the input signal has been received. This would provide a more tactile way for the user to interact with the device. The feedback device 140 may include a vibration actuation mechanism that can be embedded in the device 102 or clipped on after the fact. This can provide feedback to the user that the software has registered the input. Instead of vibration, temperature could be used (hot/cold feedback) using heaters or flow of coolant. This means the device or a clipped on structure may heat up or cool down in as feedback that the input has been received.

In another embodiment, acoustic feedback (140) may be employed. Sounds may be employed to indicate when a given trigger location has been activated. The sound may also be used to identify a trigger location. In other words, by bending the fiber a sound may be output from the system (e.g., at interface 120) or at feedback device 140. The pitch or tone of the sound may change depending on where the bend has been made. Similarly, the pitch and tone of the sound may change when the input has been received.

In another embodiment, active optical feedback (e.g., LED indicators) may be employed. The optical feedback can indicate when a given trigger location has been activated. LED indicators could be embedded in the device 102 (or input device 106). This could be done to indicate which device is active, or to indicate that the software has registered the input. The active optical feedback could indicate which bands/regions of the device are available for input and what state the input location is in. This permits more advanced interaction with the system based on the action within the current state (as indicated visually or mechanically). With high spatial resolution of LED lights complex information can be conveyed such as quantitative values or a ruler banding 318 for measuring pullback as depicted in FIG. 3E. The ruler banding 318 may be permanent color banding or LED-activated.

The LEDs could match the color in the visualization of the device rendered on the display 118. The LEDs 210 could alternatively be integrated into the launch fixture or launch base 132 (FIG. 2).

The triggering input can be used for multiple features in the software. These may include, e.g., blinking or color change of the visualization of the device on a display (118) that was triggered. This is to identify a certain device on the display screen. Designation of the ‘active’ device can be relevant for registration (register all other devices to the active device); for imaging that may be automatically following the active device (an ultrasound probe following the position of the device); for zooming in the visualization onto the distal region of the proximal device.

The triggering input from the input device 106 may be employed to save a current shape of the triggered device in memory. The triggering input may be employed to place a target based on a current shape of the device. The triggering input may be employed as input to a robotic actuation/positioning/deployment of the device. For example, once triggered a robot moves the device into a set position.

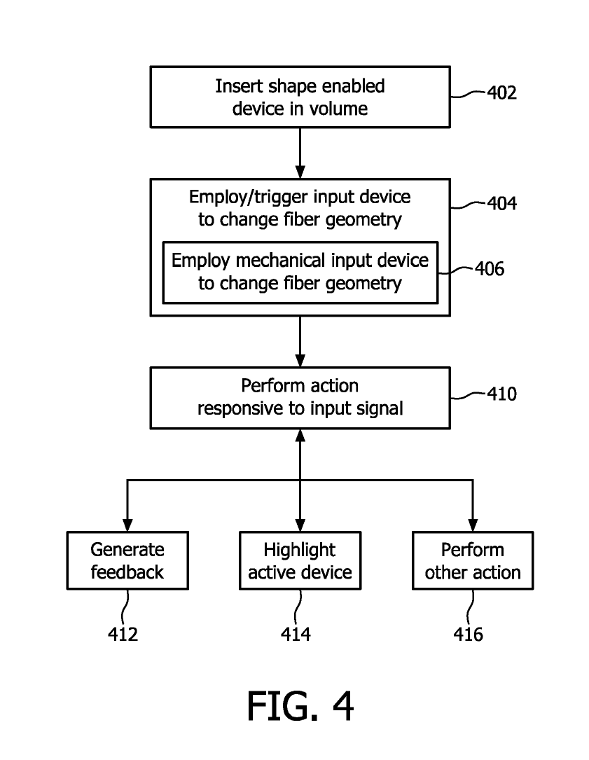

Referring to FIG. 4, a block/flow diagram showing a method for generating a manual input on a shape sensing fiber is shown in accordance with the present principles. In block 402, one or more shape enabled devices including one or more shape sensing optical fibers is inserted into a volume. This may be as part of a medical procedure or may be for mapping an internal space or cavity. In block 404, an input device is employed to change an aspect of the one or more optical fibers to generate an input signal. The aspect may be a strain due to temperature, a geometry change (bend or twist), vibratory, etc. The input device may be configured on a portion of the one or more shape sensing optical fibers. The change in geometry is distinguishable from other shape sensing data. In this way, the input signal is recognized as an input. The input device may include one or more of colored bands, textured bands, and/or bands of different stiffness to designate a region of the one or more shape sensing optical fibers where the input signal is generated. In block 406, a mechanism may be configured to generate the change in the geometry of the one or more shape sensing optical fibers distinguishable from other shape sensing data.

In block 410, an action responsive to the input signal is performed. This may include generating feedback that the input signal has been recognized in block 412, highlighting an active device or a display in block 414 and/or may include another action in block 416. In block 412, feedback is generated to indicate to a user when the input signal has been received. The feedback may include one or more of acoustic, vibratory, visual (on display or active optical feedback) and temperature feedback.

In block 414, the action may include one or more of blinking or color change of the visualization of the device (e.g., in a display or on the device (102 itself). In block 416, other actions may include: saving a current shape of the triggered device, placing a target based on the current shape of the device, inputting a robotic actuation/positioning/deployment of the device, designating an active device, registering other devices to the active device; imaging relative to the active device, zooming in on the device, etc. In one embodiment, the action may include rendering a representation of each of a plurality of shape enabled devices on a display and activating an input device of an active shape enabled device to distinguish the active shape enabled device from other shape enabled device on the display. Other actions are also contemplated.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for features for optical shape sense enabled device identification (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A system for generating a manual input on a shape sensing fiber, comprising:
a shape enabled device including:
one or more shape sensing optical fibers; and
an input device configured on a portion of the one or more shape sensing optical fibers, wherein a change in optical shape sensing data associated with the input device distinguishable from other shape sensing data, generates an input signal indicative of a triggering of a change in the input device; and
a processor system configured to receive the input signal and perform an action responsive to the input signal.

2. The system as recited in claim 1, wherein the shape enabled device includes a medical device.

3. The system as recited in claim 1, wherein the input device includes one or more colored bands to designate a region of the one or more shape sensing optical fibers where the input signal is generated.

4. The system as recited in claim 1, wherein the input device includes one or more textured bands to designate a region of the one or more shape sensing optical fibers where the input signal is generated.

5. The system as recited in claim 1, wherein the input device includes one or more bands of different stiffness to designate a region of the one or more shape sensing optical fibers where the input signal is generated.

6. The system as recited in claim 1, wherein the input device includes a mechanism configured to generate the change of the one or more shape sensing optical fibers distinguishable from other shape sensing data.

7. The system as recited in claim 1, further comprising:
a plurality of shape enabled devices each including an input device; and
a display for rendering a representation of each shape enabled device thereon, such that activating the input device of an active shape enabled device causes an effect to distinguish the active shape enabled device from other shape enabled device on the display.

8. The system as recited in claim 1, further comprising a feedback device to indicate to a user when the input signal has been received.

9. The system as recited in claim 8, wherein the feedback device includes one or more of acoustic, vibratory, visual and temperature feedback.

10. A system for generating a manual input on a shape sensing fiber, comprising:
a shape enabled device including:
one or more shape sensing optical fibers; and
an input device configured on a portion of the one or more shape sensing optical fibers;
a processor;
memory coupled to the processor, the memory including:
an optical sensing module configured to interpret optical signals from one or more shape sensing optical fibers, the optical signals including shape sensing data and an input signal generated by a user by changing the one or more shape sensing optical fibers;
wherein the input device is configured to cause a change in optical shape sensing data associated with the input device which is distinguishable from other shape sensing data, to generate the input signal indicative of a triggering of a change in the input device; and
wherein the processor and memory being configured to receive the input signal and perform an action responsive to the input signal.

11. The system as recited in claim 10, wherein the input device includes one or more colored bands to designate a region of the one or more shape sensing optical fibers where the input signal is generated.

12. The system as recited in claim 10, wherein the input device includes one or more textured bands to designate a region of the one or more shape sensing optical fibers where the input signal is generated.

13. The system as recited in claim 10, wherein the input device includes one or more bands of different stiffness to designate a region of the one or more shape sensing optical fibers where the input signal is generated.

14. The system as recited in claim 10, wherein the input device includes a mechanism configured to the change of the one or more shape sensing optical fibers distinguishable from other shape sensing data.

15. The system as recited in claim 10, further comprising:
a plurality of shape enabled devices each including an input device; and
a display for rendering a representation of each shape enabled device thereon, such that activating the input device of an active shape enabled device causes an effect to distinguish the active shape enabled device from other shape enabled device on the display.

16. (canceled)

17. (canceled)

18. (canceled)

19. (canceled)

20. (canceled)

21. (canceled)

22. (canceled)

23. (canceled)
